(19) United States
(12) Patent Application Publication
(10) Pub. No.: US 2003/0096412 A1
(43) Pub. Date: May 22, 2003

(54) Method of preparing biological materials for cryopreservation using pre-chilled protectant

(76) Inventors: Allan J. Cassell, West Heidelberg (AU); Brian Wood, Lubbock, TX (US); Samuel D. Prien, Shallowater, TX (US)

Correspondence Address: SIMON, GALASSO & FRANTZ, PLC., P.O. Box 26503, Austin, TX, 78755-0503, US

(21) Appl. No.: 09/989,715

(22) Filed: Nov. 20, 2001

(51) Int. Cl. C12N 1/04; C12N 5/00; C12N 5/02

(52) U.S. Cl. 435/374; 435/325; 435/260

(57) ABSTRACT

A method of preparing biological materials for cryopreservation is presented. The method lessens the amount of heat released by a cryoprotectant during a latent heat phase by freezing the protectant, thawing the protectant, and treating biologically active materials with the thawed protectant. First, the protectant is frozen to induce an irreversible phase change, along with an irreversible release of energy. After this phase change has occurred, the protectant is thawed and used to treat viable cells or other biologically active material about to undergo freezing. The thawed protectant within the biologically active cells has a reduced endothermic reaction upon subsequent freezing. In one embodiment, freezing the biological material includes immersing the biological material in cooling fluid and circulating the cooling fluid past the biological material at a substantially constant, pre-determined velocity and temperature such that the biological material is vitrified, thus minimizing the formation of stress fractures in cell membranes. The protectant may be propylene glycol, glycerol, DMSO, or other suitable protectants.

### FIELD OF THE INVENTION

The present invention relates generally to cryogenic preservation, and more particularly to cryopreservation processes employing protectants.

### BACKGROUND OF THE INVENTION

The use of cryopreservation to preserve cells has been known since the eighteenth century, when experiments with canine spermatozoa established that cells could be frozen and later thawed, with subsequent return of normal physiological function of a small percentage of the spermatozoa. In the early twentieth century, it was discovered that cellular recovery rates could be improved if cells were chemically prepared to withstand the freezing and thawing cycles by use of compounds collectively called cryoprotectants. The latter portion of the twentieth century saw substantial research devoted to the development of cryoprotective agents, as well as to the optimization of cooling temperatures and cooling rates for various types of cells. Today, however, even with these advances in technology, cell recovery rates from cryopreservation are often 50% or less.

Generally, cryoprotectants are composed of water, salts, sugars, a protein source, and a chemical compound termed the cryoprotectant or protectant chemical. Salts serve as buffering agents for maintaining pH within the tolerance limits of the cells or molecules to be frozen, while sugars serve as energy sources and osmotic agents. Proteins chemically stabilize cellular membrane structures before freezing to prevent activation of shock proteins.

There are various cryoprotectants in use today, for example, dimethyl sulfoxide (DMSO), propanediol (PPO), and egg-yolk/glycerol solutions. The widely accepted industry standard cryoprotectant used in the cryopreservation of most cell types is DMSO. This can be attributed to the widespread experience and knowledge of DMSO-based cryopreservation solutions, and the general perception that DMSO removes water within cellular spaces such that ice crystal formation during freezing is decreased, and thus provides superior protection and maximal cell viability.

Research efforts to date on improving cryopreservation recovery rates have generally focused on new cryoprotectants and cooling techniques. Both efforts are directed towards reduction of cellular damage which occurs when water within cells expands due to ice crystal formation during the freezing process. In theory, very slow or fast freezing rates will reduce or eliminate the formation of ice crystals within a cell. Mechanisms for very slow rates of freezing include controlled descent through nitrogen vapors into liquid nitrogen, or moving samples through super-cooled compounds, followed by plunging into liquid nitrogen. A fast freezing technique plunges cells directly into liquid nitrogen to attempt to freeze the water within the cells so rapidly that ice crystal formation is inhibited. Such an extreme drop in temperature over such a short time span often results in stress fractures within the cell membrane, thus recovery rates are adversely affected.

During the freezing process, molecules of the constituent chemicals within a cryoprotectant media are forced into alignment during the freezing process. This forced alignment causes the constituent chemicals within the media to produce an endothermic reaction, which releases energy during a latent heat phase. As freezing materials undergo the latent heat phase (with attendant endothermic reaction), this released heat causes a momentary increase in the temperature of the cryoprotectant. This latent heat, also known as heat of transformation, if measured during a phase transition at constant pressure (e.g., melting, boiling, sublimation), is simply the change in enthalpy. The change in enthalpy during an isobaric process is equal to the heat that is transferred when a system undergoes an infinitesimal process from an initial equilibrium state to a final equilibrium state.

Two critical cryopreservation parameters which must be optimized for maximum cell survival are cooling temperature and cooling rate. The alteration of cooling rate and temperature increase observed during a latent heat phase serves as an impediment to optimizing cell survival rates in cryopreservation processes, or in conventional freezing processes.

### SUMMARY OF THE INVENTION

Therefore, what is needed is an improved way to protect viable single cells, tissues, organs, nucleic acids, or other biologically active molecules during a cryogenic process, while avoiding some of the problems inherent in currently available methods. Accordingly, at least one embodiment of the present invention provides a method which may improve cryopreservation recovery rates by reducing the heat of sublimation in a protectant by pre-chilling the protectant to cause an irreversible phase change before treating biologically active materials with the thawed protectant.

In one embodiment, the protectant is frozen to induce an endothermic reaction. After the endothermic reaction has taken place, the protectant is thawed and used to treat biologically active cells about to undergo freezing. The thawed protectant within the biologically active cells does not react endothermically upon subsequent freezing, and thus the method as disclosed may substantially increase the number of viable cells remaining in biological material subjected to a cryopreservation process.

Another embodiment of the present invention provides a method of reducing the heat released by a cryoprotectant during cryopreservation. The method comprises treating biologically active material with a protectant which has been pre-chilled to cause on irreversible phase change in the protectant, and then freezing the treated biological material.

Another embodiment of the present invention provides a biological material having been subjected to a cryopreservation process, the cryopreservation process comprising pre-chilling a protectant until it is frozen to induce an irreversible release of energy from the protectant, thawing the protectant to a temperature convenient for use in treating biologically active material, treating the biological material with the thawed protectant, and freezing the treated biological material.

An object of at least one embodiment of the present invention is to improve the survival rate of biologically active material during a cryopreservation process.

An advantage of at least one embodiment of the present invention is that cellular viability loss rates are decreased because the cooling rate is not adversely affected by heat released by preservatives during the cryopreservation process.

### BRIEF DESCRIPTION OF THE DRAWINGS

Other objects, advantages, features and characteristics of the present invention, as well as methods, operation and functions of related elements of structure, and the combination of parts and economies of manufacture, will become apparent upon consideration of the following description and claims with reference to the accompanying drawings, all of which form a part of this specification, wherein like reference numerals designate corresponding parts in the various figures, and wherein:

FIG. 1 is a graph of temperature measurements of three cyroprotectants undergoing pre-conditioning by being subjected to rapid cooling over a short time interval according to at least one embodiment of the present invention;

FIG. 2 is a flow diagram illustrating a method according to at least one embodiment of the present invention;

FIG. 3 is a bar graph comparing experimental results of the cryopreservation method of liquid nitrogen and of the present invention against a control group according to at least one embodiment of the present invention;

FIG. 4 is a bar graph which illustrates the percentage of boar semen remaining motile after undergoing a freeze-thaw cycle according to at least one embodiment of the present disclosure; and

FIG. 5 is a cut-away side view of a chilling apparatus suitable for practicing a method according to at least one embodiment of the present invention.

### DETAILED DESCRIPTION OF THE FIGURES

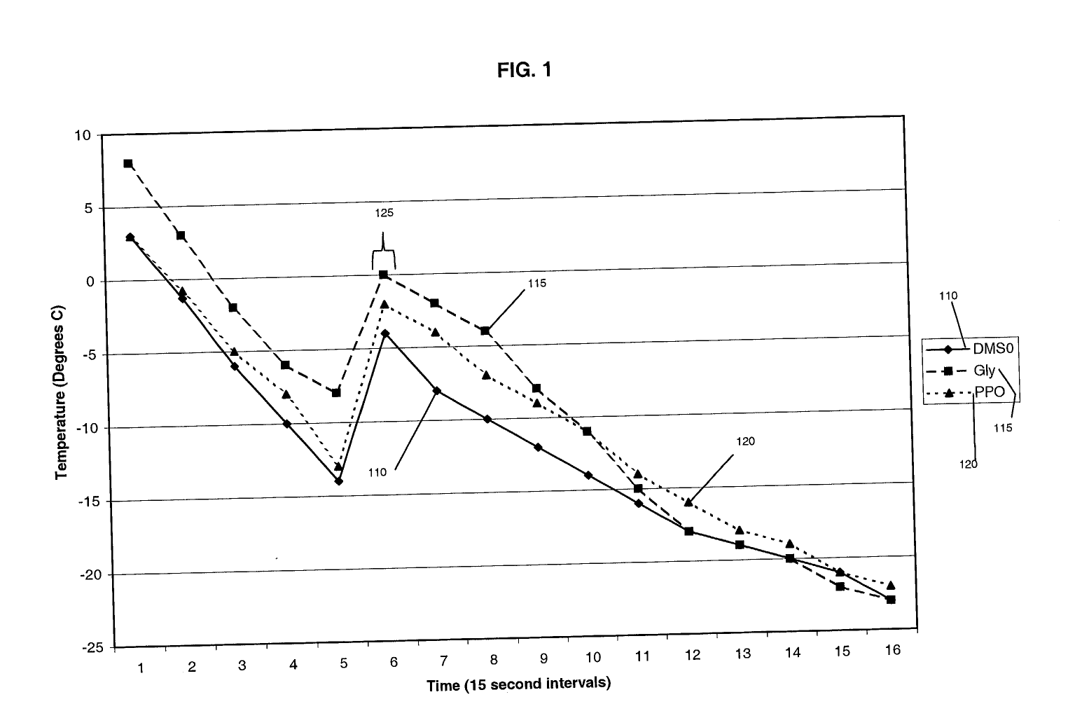

FIGS. 1-5 depict, according to various embodiments of the disclosures herein, a process for using pre-chilled protectants in cryopreservation of biologically active material, which can result in increased rates of cellular survival from the freezing process. In the various embodiments, biologically active material includes viable single cells, viable tissues, viable organs, viable nucleic acids, viable ribonucleic acids, viable amino acid based compounds and viable lipid based compounds.

In theory, most chemical reactions are bi-directional (reversible). In practice, however, many chemical reactions are found to be uni-directional (irreversible), based upon the energy requirements of a particular reaction. In the case of the protectants as embodied in the present disclosures, the release of heat during a latent heat phase is just such a unidirectional chemical reaction. Therefore, once frozen, the protectants embodied by the present disclosures exhibit a long-duration phase change capability (an irreversible phase change) upon subsequent thawing and re-freezing.

The phenomenon of the latent heat released during freezing is observed in FIG. 1, which is a graph of temperature measurements of three cyroprotectants undergoing pre-conditioning by being subjected to rapid cooling over a short time interval according to various embodiments of the present invention. The cryoprotectants measured in FIG. 1 include dimethyl sulfoxide, shown as DMSO 110, an egg-yolk/glycerol solution, shown as Gly 115, and propanediol, shown as PPO 120. The effects of the heat of transformation energy released during the cooling process are clearly seen in the measurements between time intervals 5 (at time=75 seconds) and 6 (at time=90 seconds), where a marked increase in temperature, or spike 125, is observed in all three substances. After spike 125, subsequent measurements at succeeding time intervals exhibit a decrease in temperature to the end of the measurement time period.

From a series of measurements during freezing of a solute, it is possible to determine the heat released during the latent heat phase as a momentary rise in temperature in the protectant media, as was seen in spike 125 in FIG. 1. However, if one first rapidly freezes (super-cools) the protectant in a pre-conditioning step as disclosed herein, this temperature rise is not observed because the pre-conditioned protectant has undergone a change in its chemical nature which is manifested as a long-duration phase change capability. The effects of this long-duration phase change capability resulting from pre-conditioning solute have been measured and reveal that no significant amount of heat is released when pre-conditioned protectant as disclosed herein is frozen as taught herein.

In one embodiment, there are no special temperature storage requirements for the protectant after it has undergone a freeze/thaw cycle. After pre-treatment as taught herein, the protectant demonstrates a long-duration phase change capability, and may be re-used as desired, without recurrence of an undesirable temperature spike during the freezing process. Reduction of the temperature spike according to embodiments of the present invention should increase cellular and molecular survivability and viability following the cryopreservation process.

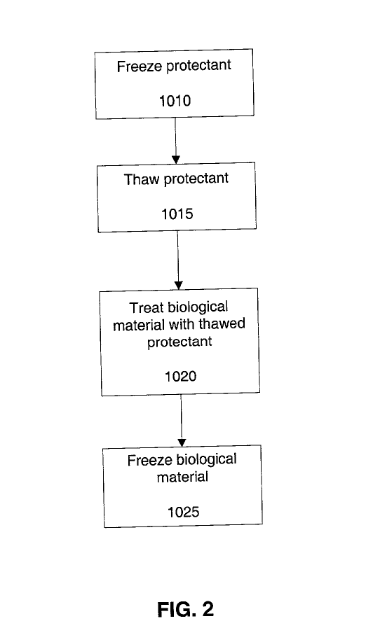

Referring now to FIG. 2, a method according to an embodiment of the present invention is illustrated. The illustrated method commences at step 1010, where a protectant is rapidly frozen to cause an irreversible release of energy (an irreversible phase change) as previously discussed. The protectants used in the various embodiments may include, but are not limited to, the following: glycerol, DMSO, or propylene glycol. In step 1015, the protectant is returned to its pre-chilled consistency by thawing the protectant to a temperature above 0 degrees Celsius. There is no separation of fluid layers upon rapidly freezing the protectant to −18 degrees Celsius or more once thawed. The lack of fluid layer separation is advantageous, as solubilization of the protectant in subsequent cooling cycles increases after a first cooling and thawing cycle. After the protectant thaws to sufficient consistency, biological materials to be frozen are imbued with the thawed protectant in preparation for freezing of the biological material, as in step 1020. In step 1025, the protectant-imbued biological materials are rapidly frozen. In an embodiment, the biological materials to which the method may be applied include biologically active material such as viable single cells, viable tissues, viable organs, viable nucleic acids, viable ribonucleic acids, viable amino acid based compounds and viable lipid based compounds.

Alternately, certain biological materials may require other chemical preparation prior to freezing. For example, chemically preparing the material may include pretreatment of the material with agents (stabilizers) that increase cellular viability by removing harmful substances secreted by the cells during growth or cell death. Useful stabilizers include those chemicals and chemical compounds, many of which are known to those skilled in the art, which sequester highly reactive and damaging molecules such as oxygen radicals.

The steps illustrated in FIG. 2 are shown and discussed in a sequential order. However, the illustrated method is of a nature wherein some or all of the steps are continuously performed, and may be performed in a different order. For example, if a batch of the protectant is on hand which has already undergone a freeze/thaw cycle, it is not necessary to re-freeze the protectant prior to treating biological material with it.

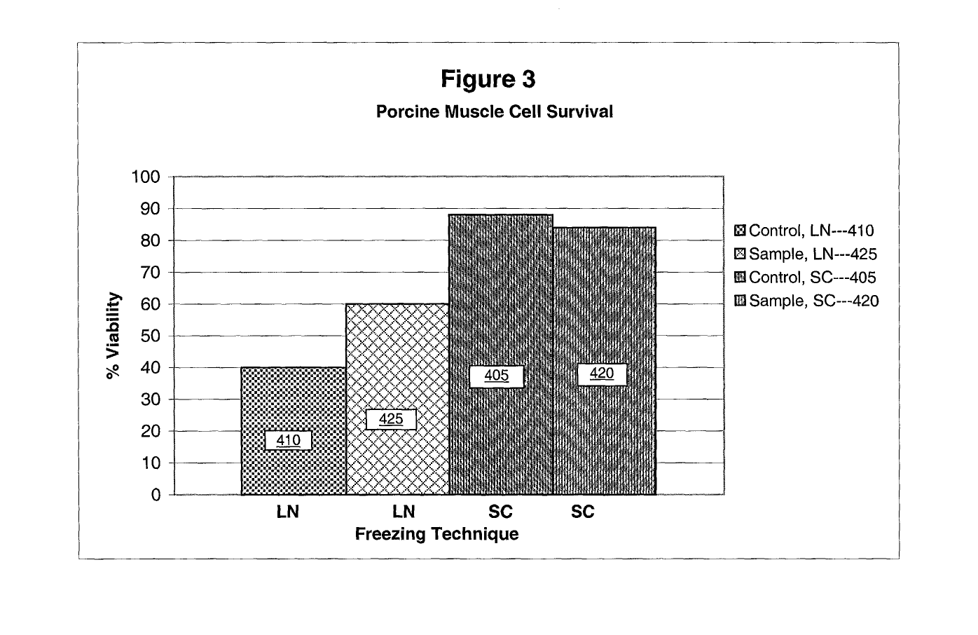

Studies conducted utilizing the techniques disclosed herein indicate that improvement in cellular viability of 40% or more may be obtained. Results of experiments with porcine muscle cells are presented in FIG. 3, a bar graph presenting results of the cryopreservation method of liquid nitrogen (LN) and an embodiment of the present invention (SC) against a control group, referred to as bar graph 400. Bar graph 400 compares the number of viable porcine muscle cells which have undergone cryopreservation with a pre-chilled protectant against a control group. Controls were subjected to cryopreservation with a protectant which had not been pre-chilled according to the various embodiments disclosed herein.

The control group without pre-chilled protectant 405 was frozen to about minus −25° Celsius, according to a high temperature freezing method as disclosed herein, while the control group without pre-chilled protectant 410 underwent freezing in liquid nitrogen (LN) to about minus 196° Celsius. Both control groups 405 and 410, and pre-chilled protectant treated groups 420 and 425 were taken from a common tissue source, which was divided into multiple groups in order to be subjected to the different treatments and freezing techniques (LN and SC). Porcine muscle group 425 was subjected to cryopreservation with liquid nitrogen (LN) after treatment with a pre-chilled protectant as disclosed herein. Porcine muscle group 420 was subjected to cryopreservation with the same high temperature freezing method used to freeze control group 405, after treatment with a pre-chilled protectant as disclosed herein.

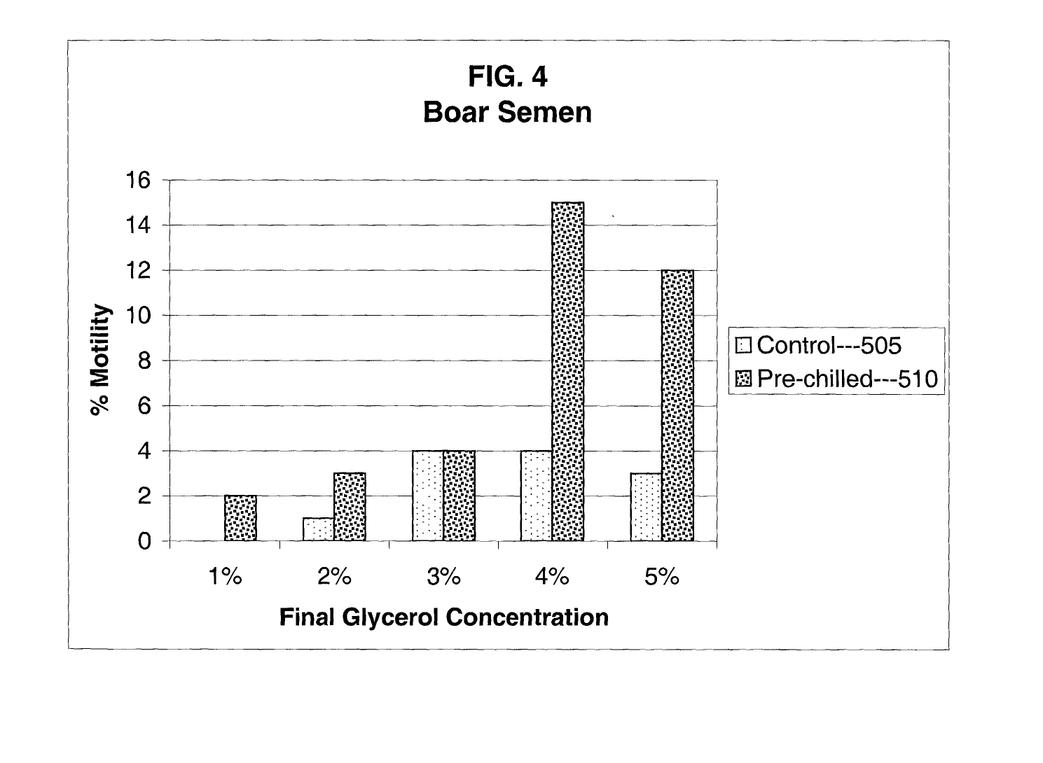

As bar graph 400 shows, with the liquid nitrogen (LN) technique, pre-chilled group 425 exhibited a percentage viability rate after thawing of between about 60-70%, while the control group without pre-chilled protectant 410 using the same LN freezing technique exhibited a percent viability rate after thawing of between 40-50%. With the freezing technique of a high-temperature freezing method as disclosed herein, pre-chilled group 420 exhibited a percent viability rate of between about 80-90%, while the control group without pre-chilled protectant 405 exhibited a percent viability rate of between about 80-90%. In terms of overall cellular survivability, the LN cryopreservation technique is considerably less than that of the high-temperature freezing method. In the case of groups 410 and 425, subjected to the freezing technique of liquid nitrogen, the use of pre-chilled protectants for cells as disclosed herein results in a noticeable increase in survivability over cells treated with a protectant that had not been pre-chilled. However, the high-temperature freezing method results in an increase in cell survival that is nearly double that of the LN technique, as comparison of pre-chilled protectant treated groups 420 and 425 reveals. Although the use of pre-chilled protectants did not significantly change the survival rate when the same cells were frozen with the high-temperature freezing method, for example groups 405 and 420, other cell types (such as the boar spermatozoa presented in FIG. 4) from separate experiments showed significant improvement in both freezing techniques (LN and SC).

Referring now to FIG. 4, a graph illustrating the percent motility of boar semen in samples treated with conventional protectants and samples treated with pre-chilled protectants according to an embodiment of the present disclosure, after the samples have undergone cryopreservation (freezing) and thaw for examination. The protectants used for this study were glycerol and water mixtures with varying concentrations of glycerol as a percent of weight. Hence the numbers 1%, 2%, et cetera on the ordinate indicate a1%, 2%, 3%, 4%, or 5% final glycerol concentration. The control group 505 indicates the samples of semen which were treated with protectants of varying concentrations (1% -5% by weight) of glycerol that had not been subjected to pre-chilling according to the embodiments disclosed herein. The pre-chilled group 510 indicates the samples of semen which were treated with protectants of various concentrations of glycerol which had been subjected to pre-chilling as disclosed herein. The high-temperature freezing method as embodied herein was employed to freeze the various boar semen samples.

As seen in FIG. 4, the pre-chilled groups 510 exhibited a higher percentage motility than the control groups 505 for all glycerol concentrations, with the exception of the 3% glycerol data points, which are approximately equal. These data suggest that cells more sensitive to freezing may show superior survival rates if frozen with media which has been pre-treated to exhibit a long-duration phase change capability (an irreversible phase change) as disclosed herein. Study results further suggest that the techniques disclosed herein may be applied to cryopreservation of biological material from species which were previously considered resistant to these technologies, as well as to all other mammalian species. In addition to spermatozoa, there are numerous other fields such as skin, cell lines, proteins and other biologically active materials which could also benefit from application of the method as disclosed.

In one embodiment, application of the method as disclosed can be extended to humans to provide lower cost infertility treatments in the area of artificial insemination or in vitro fertilization. Because some of the cryoprotectants disclosed herein, such as propylene glycol, do not exhibit the toxicity effects of some other cryoprotectants, such as DMSO, there should be no side effects from use of the long-duration phase change protectants in those patients into whom protectant-treated sperm is introduced.

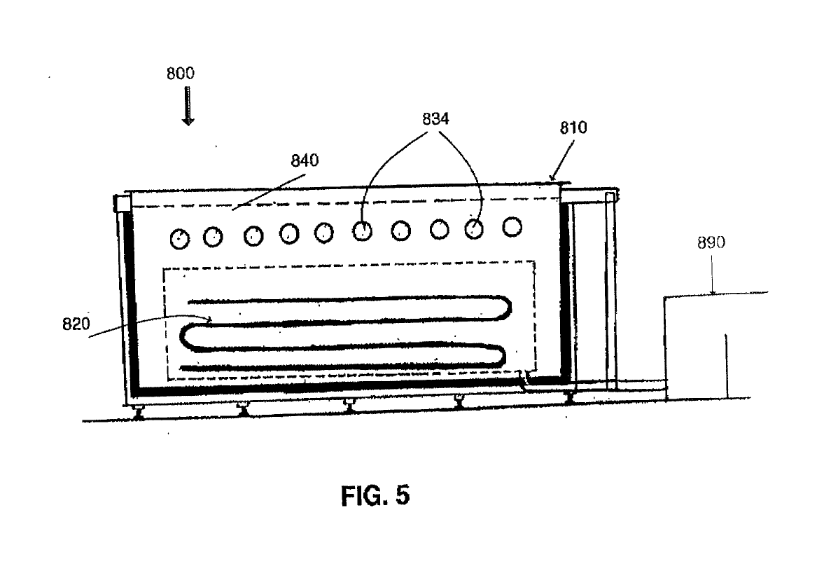

Referring next to FIG. 5, a chilling apparatus suitable for use with the method is illustrated according to at least one embodiment of the present invention, and designated generally as cooling unit 800. Cooling unit 800 preferably comprises tank 810 containing cooling fluid 840. Submersed in cooling fluid 840 are circulation mechanisms 834, such as motor and impeller combinations, and heat exchanging coil 820. Material to be chilled may include, but is not limited to, viable single cells, tissues, organs, nucleic acids, ribonucleic acids, amino acid based compounds, lipid based compounds, and other biologically active molecules. External to tank 810, and coupled to heat exchanging coil 820, is refrigeration unit 890.

Tank 810 may be of any dimensions necessary to immerse material to be frozen in a volume of cooling fluid 840, in which the dimensions are scaled multiples of 12 inches by 24 inches by 48 inches. Other size tanks may be employed consistent with the teachings set forth herein. For example, in one embodiment (not illustrated), tank 810 is sized to hold just enough cooling fluid 840, so containers can be placed in tank 810 for rapid freezing of suspensions including biological materials and cryoprotectants. In other embodiments, tank 810 is large enough to completely immerse entire organisms for rapid freezing. It will be appreciated that tank 810 can be made larger or smaller, as needed, to efficiently accommodate various sizes and quantities of material to be frozen.

Tank 810 holds cooling fluid 840. In one embodiment, the cooling fluid is a food-grade solute. Good examples of food-grade quality fluids are those based on propylene glycol, sodium chloride solutions, glycerol, or the like. In a preferred embodiment, the cooling fluid is the protectant propylene glycol. While various containers may be used to hold the biological material, some embodiments of the present invention provide for the biological material to be directly immersed in the cooling fluid for rapid and effective freezing.

In order to freeze material while avoiding the formation of ice crystals, one embodiment of the present invention circulates cooling fluid 840 past the material to be frozen, at a relatively constant rate of 35 liters per minute for every foot of cooling fluid contained in an area not more than 24 inches wide by 48 inches deep. The necessary circulation is provided by one or more circulation mechanisms 834 for example, a motor and impeller combination. In at least one embodiment of the present invention, submersed circulation mechanisms 834 circulate cooling fluid 840 past material to be frozen. Other circulation mechanisms 834, including various pumps (not illustrated), can be employed consistent with the objects of the present invention. At least one embodiment of the present invention increases the area and volume through which cooling fluid is circulated by employing at least one circulation mechanism 834. In embodiments using multiple circulation mechanisms 834, the area and volume of cooling fluid circulation are increased in direct proportion to each additional circulation mechanism employed. For example, in a preferred embodiment, one additional circulation mechanism is used for each foot of cooling fluid that is to be circulated through an area of not more than about 24 inches wide by 48 inches deep.

Preferably, motors within circulation mechanism 834 can be controlled to maintain a constant predetermined velocity of cooling fluid flow past the materials to be preserved, while at the same time maintaining an even distribution of cooling fluid temperature to within +/−0.5 degrees Celsius at all points within tank 810. The substantially constant predetermined velocity of cooling fluid circulating past the material or product provides a constant, measured removal of heat, which allows for the chilling or freezing of the material. In one embodiment, cooling fluid properties, such as viscosity, temperature, etc., are measured and processed, and control signals are sent to circulation mechanism 834 such that the motor within circulation mechanism 834 can increase or decrease the rotational speed or torque of impellers as needed. In other embodiments, motors are constructed to maintain a given rotational velocity over a range of fluid conditions without producing additional heat. In such a case, the torque or rotational speed of impellers imparted by motors are not externally controlled. Of note is the fact that no external pumps, shafts, or pulleys are needed in the chilling apparatus. Combination motors and impellers, or other circulation mechanisms 834, are immersed directly in cooling fluid 840. As a result, cooling fluid 840 not only freezes material placed in tank 810, but cooling fluid 840 also provides cooling for components (i.e., motors and impellers) within circulation mechanisms 834.

Heat exchanging coil 820 is preferably a “multi-path coil,” which allows refrigerant to travel through multiple paths (i.e. three or more paths), in contrast to conventional refrigeration coils in which refrigerant is generally restricted to one or two continuous paths. In addition, the coil size is in direct relationship to the cross sectional area containing the measured amount of the cooling fluid 840. For example, in a preferred embodiment, tank 810 is one foot long, two feet deep and four feet wide, and uses a heat exchanging coil 820 that is one foot by two feet. If the length of tank 810 is increased to twenty feet, then the length of heat exchanging coil 820 is also increased to twenty feet. As a result, heat exchanging coil 820 can be made approximately fifty percent of the size of a conventional coil required to handle the same heat load. Circulation mechanisms 834 circulate chilled cooling fluid 840 over material to be frozen, and then transport warmer cooling fluid to heat exchanging coil 820, which is submersed in cooling fluid 840. In at least one embodiment, heat exchanging coil 820 is so designed to remove not less than the same amount of heat from cooling fluid 840 as that removed from the material being frozen, thereby maintaining the temperature of cooling fluid 840 in a predetermined range. Heat exchanging coil 820 is connected to refrigeration unit 890, which removes the heat from heat exchanging coil 820 and the system.

In a preferred embodiment, refrigeration unit 890 is designed to match the load requirement of heat exchanging coil 820, so that heat is removed from the system in a balanced and efficient manner, resulting in the controlled, rapid freezing of a material. The efficiency of the refrigeration unit 890 is directly related to the method employed for controlling suction pressures by the efficient feeding of the heat exchange coil 820 and the efficient output of compressors used in refrigeration unit 890.

This methodology requires very close tolerances to be maintained between the refrigerant and cooling fluid 840 temperatures, and between the condensing temperature and the ambient temperature. These temperature criteria, together with the design of the heat exchange coil 820, allows heat exchange coil 820 to be fed more efficiently, which in turn allows the compressor to be fed in a balanced and tightly controlled manner to achieve in excess of twenty-five percent greater performance from the compressors than that which is accepted as the compressor manufacturer's standard rating.

Note that in the embodiment illustrated in FIG. 5, refrigeration unit 890 is an external, remotely located refrigeration system. However, in another embodiment (not illustrated), refrigeration unit 890 is incorporated into another section of tank 810. It will be appreciated that various configurations for refrigeration unit 890 may be more or less appropriate for certain configurations of cooling unit 800. For example, if tank 810 is extremely large, a separate refrigeration unit 890 may be desirable, while a portable embodiment may benefit from an integrated refrigeration unit 890. Such an integration is only made possible by the efficiencies achieved by implementing the principles as set forth herein, and particularly the use of a reduced-size heat exchanging coil.

By virtue of refrigeration unit 890 and heat exchanging coil 820, in a preferred embodiment, the cooling fluid is cooled to a temperature of between −20° Celsius and −30° Celsius, with a temperature differential throughout the cooling fluid of less than about +/−0.50 Celsius. In other embodiments, the cooling fluid is cooled to temperatures outside the −20° Celsius to −30° Celsius range in order to control the rate at which a substance is to be frozen. Other embodiments control the circulation rate of the cooling fluid to achieve desired freezing rates. Alternatively, the volume of cooling fluid may be changed in order to facilitate a particular freezing rate. It will be appreciated that various combinations of cooling fluid circulation rate, cooling fluid volume, and cooling fluid temperature can be used to achieve desired freezing rates.

In the preceding detailed description, reference has been made to the accompanying drawings which form a part hereof, and in which are shown by way of illustration specific embodiments in which the invention may be practiced. These embodiments have been described in sufficient detail to enable those skilled in the art to practice the invention, and it is to be understood that other embodiments may be utilized and that logical, mechanical, chemical and electrical changes may be made without departing from the spirit or scope of the invention. To avoid detail not necessary to enable those skilled in the art to practice the invention, the description omits certain information known to those skilled in the art. The preceding detailed description is, therefore, not to be taken in a limiting sense, and the scope of the present invention is defined only be th appended claims.

What is claimed is:

1. A method comprising the steps of:
chilling a protectant to cause an irreversible release of energy from the protectant;
treating biologically active material with the protectant; and
freezing the treated biologically active material.

2. The method as in claim 1, wherein said pre-conditioned solute is a solute having been conditioned by being super-cooled at an average rate of at least about 6.5° C. per minute.

3. The method as in claim 1, wherein said pre-conditioned solute is a solute having been conditioned by being super-cooled from room temperature to a temperature of less than about −23° C.

4. The method as in claim 1, wherein said pre-conditioned solute is a solute having been conditioned by being super-cooled from room temperature to between about −23° C. and −26° C.

5. The method as in claim 1, wherein said pre-conditioned solute is a solute having been conditioned by being super-cooled at an average rate of between about 6.5° C. and 8.5° C. per minute.

6. The method as in claim 1, wherein said pre-conditioned solute is a solute having been conditioned by being super-cooled, for at least a portion of time, at an average rate of at least about 17° C. per minute.

7. The system as in claim 1, wherein the heat absorption rate of the pre-conditioned solute is about 135 BTU at a temperature of between about −23° C. and −26° C.

8. The method as in claim 1, further including the step of warming the protectant prior to the step of treating the biologically active material.

9. The method as in claim 8, wherein the step of warming the protectant includes warming the protectant to above 0 degrees Celsius.

10. The method as in claim 1, wherein the protectant includes propylene glycol.

11. The method as in claim 1, wherein the protectant includes glycerol.

12. The method as in claim 1, wherein the protectant includes DMSO.

13. The method as in claim 1, wherein the biologically active material includes:
viable single cells, viable tissues, viable organs, viable nucleic acids, viable ribonucleic acids, viable amino acid based compounds and viable lipid based compounds.

14. A method comprising the steps of:
chilling a protectant to below about −23 degrees Celsius to cause an irreversible release of energy from the protectant;
warming the protectant to above 0 degrees Celsius;
treating biologically active material with the protectant; and
freezing the treated biologically active material.

15. The method as in claim 14, wherein said pre-conditioned solute is a solute having been conditioned by being super-cooled at an average rate of at least about 6.5° C. per minute.

16. The method as in claim 14, wherein said pre-conditioned solute is a solute having been conditioned by being super-cooled from room temperature to a temperature of less than about −23° C.

17. The method as in claim 14, wherein said pre-conditioned solute is a solute having been conditioned by being super-cooled from room temperature to between about −23° C. and −26° C.

18. The method as in claim 14, wherein said pre-conditioned solute is a solute having been conditioned by being super-cooled at an average rate of between about 6.5° C. and 8.5° C. per minute.

19. The method as in claim 14, wherein said pre-conditioned solute is a solute having been conditioned by being super-cooled, for at least a portion of time, at an average rate of at least about 17° C. per minute.

20. The method as in claim 14, wherein the heat absorption rate of the pre-conditioned solute is about 135 BTU at a temperature of between about −23° C. and −26° C.

21. The method as in claim 14, wherein the protectant includes propylene glycol.

22. The method as in claim 14, wherein the protectant includes glycerol.

23. The method as in claim 14, wherein the protectant includes DMSO.

24. The method as in claim 14, wherein the biologically active material includes:
viable single cells, viable tissues, viable organs, viable nucleic acids, viable ribonucleic acids, viable amino acid based compounds and viable lipid based compounds.

25. A biological material having been subjected to a cryopreservation process, the cryopreservation process comprising:
chilling a protectant to cause an irreversible release of energy from the protectant;
treating biologically active material with the protectant; and
freezing the treated biologically active material.

26. The biological material as in claim 25, wherein said pre-conditioned solute is a solute having been conditioned by being super-cooled at an average rate of at least about 6.5° C. per minute.

27. The biological material as in claim 25, wherein said pre-conditioned solute is a solute having been conditioned by being super-cooled from room temperature to a temperature of less than about −23° C.

28. The biological material as in claim 25, wherein said pre-conditioned solute is a solute having been conditioned by being super-cooled from room temperature to between about −23° C. and −26° C.

29. The biological material as in claim 25, wherein said pre-conditioned solute is a solute having been conditioned by being super-cooled at an average rate of between about 6.5° C. and 8.5° C. per minute.

30. The biological material as in claim 25, wherein said pre-conditioned solute is a solute having been conditioned by being super-cooled, for at least a portion of time, at an average rate of at least about 17° C. per minute.

31. The biological material as in claim 25, wherein the heat absorption rate of the pre-conditioned solute is about 135 BTU at a temperature of between about −23° C. and −26° C.

32. The biological material as in claim 25, wherein the cryopreservation process includes warming the protectant prior to the step of treating the biologically active material.

33. The biological material as in claim 32, wherein the cryopreservation process includes warming the protectant to above 0 degrees Celsius.

34. The biological material as in claim 25, wherein said biological material comprises viable single cells.

35. The biological material as in claim 25, wherein said biological material comprises viable tissues.

36. The biological material as in claim 25, wherein said biological material comprises viable organs.

37. The biological material as in claim 25, wherein said biological material comprises viable nucleic acids.

38. The biological material as in claim 25, wherein the biological material comprises viable ribonucleic acids.

39. The biological material as in claim 25, wherein the biological material comprises viable amino acid based compounds.

40. The biological material as in claim 25, wherein the biological material comprises viable lipid based compounds.
